(19) United States
(12) Patent Application Publication
(10) Pub. No.: US 2008/0033455 A1
(43) Pub. Date: Feb. 7, 2008

(54) HAIR EXTRACTION DEVICE AND METHOD FOR ITS USE

(76) Inventors: William R. Rassman, (US); Jae P. Pak, (US)

Correspondence Address: William R. Rassman, M.D., Suite 301, 9911 W. Pico Blvd, Los Angeles, CA, 90035, US

(21) Appl. No.: 11/462,102

(22) Filed: Aug. 3, 2006

(51) Int. Cl. A61B 17/50

(52) U.S. Cl. 606/133

(57) ABSTRACT

The harvesting of hair for a hair transplant procedure employs a hollow drill with an imaging system which permits alignment of center of the cutting edge of a hollow tube with the axis of the follicular unit to be removed. The diameter of the hollow tube is chosen such that when properly aligned, a follicular unit is removed without damaging critical anatomical portions of the follicles. In one embodiment, a medium in either liquid or vapor form is introduced to cool or freeze the skin over the surface of the selected follicular unit. In another embodiment, suction is applied to aid in the removal of the excised follicular unit. In still another embodiment, a robotic arm is employed to position the hollow needle in each instance. In another embodiment, an electrical current or heat is applied to kill the shaft of the selected follicular unit without its removal.

### RELATE APPLICATION

The present application is related to U.S. Pat. No. 6,572625 B1, issued Jun. 3, 2003, included by reference herein.

### FIELD OF THE INVENTION

This invention relates to the isolation and or extraction of hair follicles from an area and more particularly to a device for controllably positioning an instrument for removing follicular units of hair and stabilizing the hair bearing skin and its layers.

### BACKGROUND OF THE INVENTION

Hair transplant procedures have been carried out for decades. Initially, a punch was used to remove a circular area of hairy skin containing ten or more follicular units (of 1-3 hairs each). The area of hairy skin replaced a like area of bald skin removed from the patient. Several of such “plugs,” were placed into areas in the bald part of the head.

The circular punch tool was later replaced by a hollow powered drill and the space left in the donor area was left to heal naturally. Both of these prior art procedures allowed wounds to stay open for weeks at a time exposing a patient to the discomfort from large wounds measuring 3-5 millimeters in diameter.

Today's standard procedure often referred as follicular unit extraction is done with a “punch” that is around 1 millimeters in diameter which leaves virtually no scar and promotes faster healing times due to the small size of the wound. It is a more aesthetically pleasing procedure because the transplanted hair is in its natural follicular unit grouping of 1 to 3 hairs.

The problem with today's follicular unit extraction is that it is difficult to harvest one follicular unit at a time because of the inherent variability of individual patient's skin and hair characteristics. Working with a follicular extraction tool that is about 1 mm in diameter gives a narrow margin of error for the surgeon.

Other solutions to the “punch” or drilling method of harvesting hair follicles is to excise a strip of skin containing the donor hair using a scalpel. This strip of skin is processed by mean of dividing up and separating individual hair follicles preferably under a dissecting microscope. Thus the harvesting of individual hair follicles is carried out separately outside the human body. The resulting wound from the strip of skin that was excised is closed with sutures or staples.

Shortcomings of the strip excision include longer healing time and unavoidable linear scar. This linear scar is sometimes seen as the stigmata of a hair transplant surgery.

### SUMMARY OF THE INVENTION

In accordance with the principles of the present 0invention, a hollow instrument with a cutting or dissecting edge such as a drill or punch or needle (sharp or dull) with a diameter slightly larger than a follicular unit is used to cut into the skin. The instrument includes an imaging system such as a video system which allows the health care professional to align the instrument along the axis of the follicular unit and to produce a cutting action outside of the follicular unit's critical anatomical parts. As the wound is very small, this results in relatively fast healing, less bleeding and virtually no grossly visible scar tissue formation.

The invention is based on the recognition that a follicular unit can be harvested by freezing or cooling the superficial area of the follicular unit to facilitate the harvesting process. A similar process can be employed to kill a hair follicle by inserting a needle for delivering an electric current or by delivering a high ultrasound signal to the growth centers along the hair shaft. When used as a harvesting tool, the freezing or cooling of the localized area of skin results in a superficial freeze or cooling around the targeted follicular unit, stabilizing the follicular unit in the ‘frozen skin’ facilitating the removal of the follicular unit as detailed in Pat. No. 6,572,625 B1 issued Jun. 3, 2003.

The freezing or cooling of the dermal layer surrounding a follicular unit to be excised has been found not only to be simpler to implement but also to facilitate the removal of the unit even more than the introduction of fluid under the skin as described in the above-noted patent. When used for hair destruction, it has been found advantageous to align and stabilize the shaft of the follicular unit in order to disrupt the growth center of the follicular unit, thereby killing it.

In accordance with one embodiment of this invention, the instrument of FIG. 1 is adapted to apply a liquid or air to relatively cool the follicular unit being viewed to near freezing or freezing temperature. The cooling of a follicular unit prior to excising the unit, after it is aligned, not only stabilizes the deeper dermal structures with respect to the more superficial structures, but does so in a manner to anesthetize the area and allow removal with virtually no damage to surrounding areas.

In accordance with another embodiment a multiple dimensional stabilization gantry is employed to fix the hollow instrument and thus the imaging system. Such a ,device can be done without direct human involvement, acquiring the appropriate orientation for follicular unit extraction or destruction based upon the same technology as described in the aforementioned patent. In still another embodiment a suction device is positioned to create traction and or better field of view along the follicular unit to ensure that cutting proceeds only to a prescribed path and or depth.

### BRIEF DESCRIPTION OF THE DRAWINGS

FIG. 1 is a schematic block diagram of a hair transplant harvesting device in accordance with the principles of this invention;

FIG. 2 is a schematic side view of a hand-held portion of the system of FIG. 1;

FIGS. 3 and 4 are enlarged schematic cross sectional and perspective views of a subassembly of the hand-held portion shown in FIG. 2;

FIGS. 5 and 6 are representations of video images produced by the apparatus of FIGS. 1-4; and

FIG. 7 is a flow diagram of the method practiced by the system of FIGS. 1-7, and

FIG. 8 is a schematic side view of a robotic subassembly for excising follicular units from a donor area;

### DETAILED DESCRIPTION OF ILLUSTRATIVE EMBODIMENTS OF THIS INVENTION

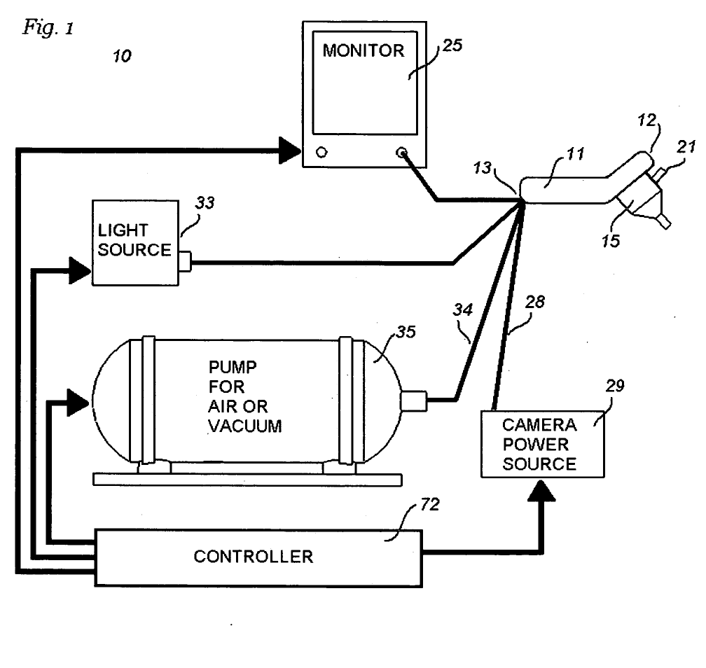

FIG. 1 is a block diagram of a system 10 in accordance with this invention. The system includes an instrument 11 having a proximal end and a distal end 12 and 13 respectively. A tubular shaped subassembly 15 extends from the proximal end of the instrument.

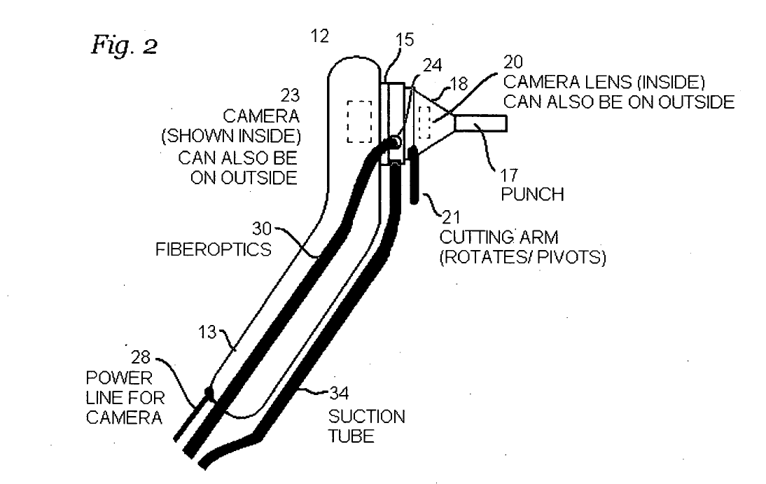

FIG. 2 is an enlarged schematic side view of instrument 11. Subassembly 15 can be seen to have a general shape similar to that of an instrument commonly used to examine the human ear. The subassembly includes a hollow needle or punch 17 and a conical section 18 which enlarges in diameter as it extends upwards and to the left as viewed in the figure.

The end of section 18 with the relatively large diameter is coupled to the cylindrical portion of the subassembly by a rotating member or lip 20. Rotating member 20 is operated by cutting arm 21 and serves to extend (or advance) hollow needle 17 when rotated.

Instrument 11 includes a solid state camera such as a charge-coupled device (CCD) camera represented by dashed rectangular line 23 shown in FIG. 2. Subassembly 15 also includes a lens 24. The camera and lens are components of an imaging system positioned to capture an image via the hollow needle and to display that image on monitor 25 of FIG. 1. Power for the CCD camera is provided via cable 28 of FIGS. 1 and 2 from power source 29 of FIG. 1.

Illumination of the field of view for the camera is supplied via optical fibers 30 extending from subassembly 15 to light source 33 of FIG. 1.

In one embodiment of the invention, a suction tube 34 also is provided. Tube 34 extends between subassembly 15 and an air pump 35 shown in FIG. 1. Suction may be provided to assist in extracting a follicular unit when arm 21 is rotated by a user to advance hollow needle 17 into the skin of a patient.

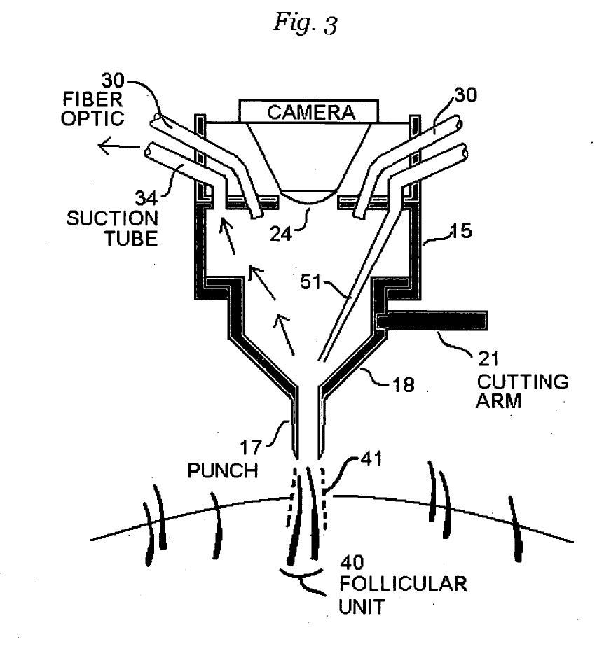

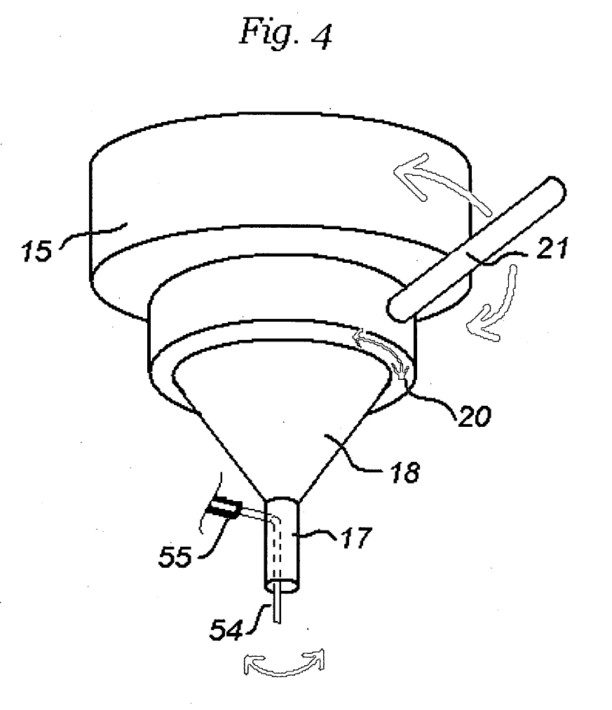

FIG. 3 shows an enlarged cross section of subassembly 15 showing the relative position of lens 24, optical fibers (30), suction tube 34, hollow needle 17, conical section 18, and rotating member 20 with cutting arm 21. FIG. 4 shows an enlarged perspective view of subassembly 15 showing hollow needle 17, conical section 18, rotating member 20 with cutting arm 21. It is clear from the views of FIGS. 3 and 4 that a user observes a follicular unit 40 as shown in FIG. 4 by moving instrument 11 (FIG. 1) until a selected follicular unit is in the center of the field of view 41. The user observes the field of view in monitor 25 of FIG. 1. The diameter of hollow needle 17 is chosen sufficiently wide to cut past beyond the critical anatomical structures of a follicular unit thus avoiding any damage to the unit.

The instrument as shown in FIG. 3 also is adapted to include a reservoir of, for example, liquid nitrogen or ethylene dioxide for freezing the follicular unit (40) in view. Tube 51 is shown in FIG. 3 to indicate a supply of the freezing material if the material is of a type to require maintenance under pressure and/or is not sufficiently transparent to avoid obstructing the viewing of the follicular unit (40).

It has also been found under certain conditions or circumstances to kill or neutralize the hair shaft of the follicular unit to be excised. The follicular unit is killed or neutralized conveniently by inserting a needle into the follicular unit and delivering an electric current for a predetermined period of time to in essence neutralize or kill the growth center of the follicular unit. FIG. 4 shows a needle 54 connected via conductor 55 for this purpose. The conductor is connected to a power source not shown. The electrical current can be initiated by the movement of arm 21 under the control of a controller represented by block 72 shown in FIG. 1.

Alternatively, needle 54 could be adapted to supply ultrasound energy to accomplish the same goal of neutralizing or killing the follicular unit.

Once the follicular unit is frozen, excising of the unit may proceed.

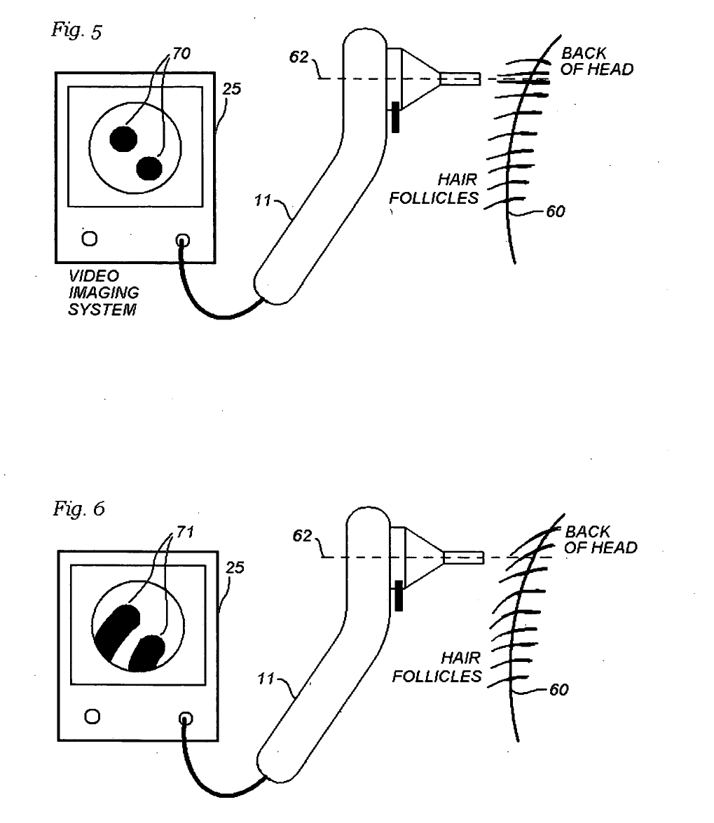

FIGS. 5 and 6 show the image of a representative follicular unit when instrument 11 is aligned with the unit and when it is not respectively. Both FIGS. 5 and 6 represent the back of a patient's head 60 with hair follicles extending downwards and to the left as viewed. In FIG. 5 instrument 11 is seen to align its axis 62 and the hair follicles are not aligned. The resulting images in monitor 25 are dots 70 and lines 71 respectively.

The various components of the system of FIG. 1 may be any components capable of operating as described. The operation of the component is controlled by a controller represented by block 72 of FIG. 1.

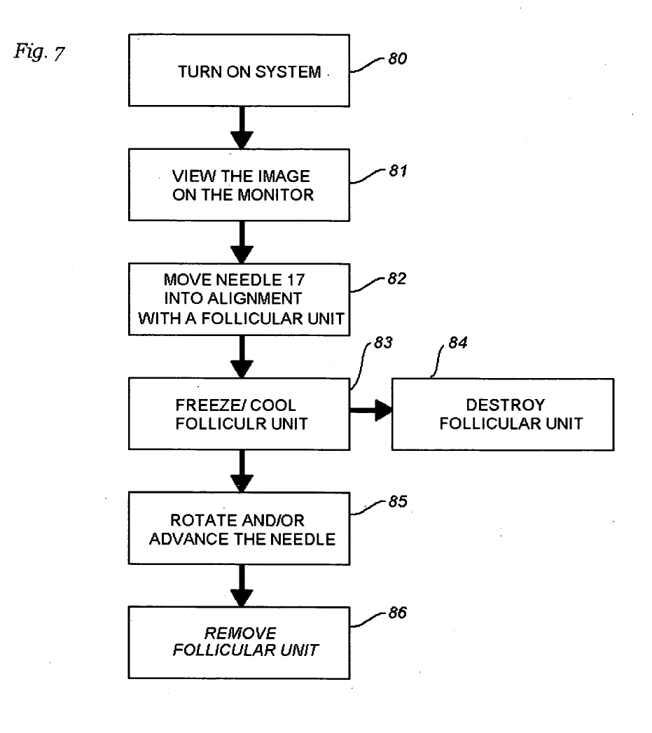

FIG. 7 is a flow diagram of the method practiced by the system of FIG. 1. The system is used with a patient having a shaved head so that the donor area of the head exhibits erect hair follicles. A user positions the hollow needle over the skin of a patient after turning on the monitor, the light source and the camera. The procedure starts by turning on the system as represented by block 80 of FIG. 7 and the image of the field of view is observed on the monitor (25) as indicated by block 81.

The user moves the instrument (11) until the field of view (illustratively through the needle) is in alignment with a follicular unit producing an image as shown in FIG. 5. This step is represented by block 82.

The instrument is now properly positioned with the top of the needle juxtaposed against the skin of a patient with a selected follicular unit in the field of view and with the axis of the needle aligned with the axis of the follicular unit. The user now administers the freezing material and then applies a current to kill the shaft of the follicular unit in view as shown by blocks 83 and 84. The user now moves arm 21 to advance the needle while rotating the cutting edge of the needle to excise the unit. The rotation of the needle and the removal of the unit are represented by blocks 85 and 86.

The removal of the excised unit is expedited by the suction tube 34 and air pump 35 of FIG. 1. But any suitable method of removal is adequate. Tweezers may be used, for example.

The spacing between adjacent follicular units varies randomly but typically is greater than one millimeter. The critical anatomical structure of a unit is only a fraction of that distance thus dictating a minimum diameter of 0.50 millimeters for the hollow needle. It is thus, convenient to secure an x/y/z gantry (not shown) to the head of a patient and to secure the “hand held” instrument so that it can be controlled by adjusting micromanipulators.

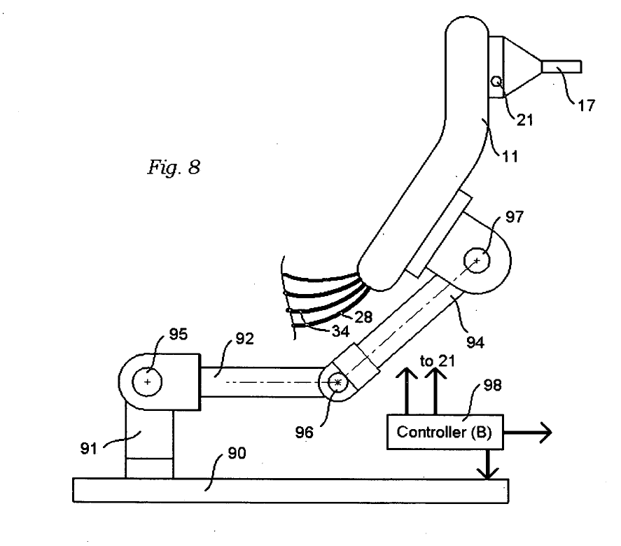

FIG. 8 is a schematic side view of a robotic subassembly for extracting follicular untis of hair from a donor area in the manner described in connection with FIGS. 1, 2, and 3. The robotic subassembly comprises of a positioning table 90 to which the instrument 11 of FIGS. 1, 2, and 3 is connected by means of Post 91. Positioning table 90 is operative to move post 91 in a X,Y,Z and rotational planes. Post 91 is operative to actuate arm 92 along and or around its axis. Instrument 11 is connected to Post 91 by arms 92 and 94 at pivot points 95, 96, and 97 which are set to adjust Punch 17 with a selected donor area.

The Operation is controlled by a controller 98 responsive to move punch 17 to a position shown in FIG. 5, to initiate a localized freezing of the follicular unit view and to advance the punch along its axis to a depth to excise or extract the follicular unit and to manipulate cutting arm 21 to facilitate the removal of the follicular unit.

The robotic subassembly of FIG. 8 may be adapted for administering a freezing or cooling step and/or a growth center destruction step as described in connection with FIGS. 3 and 4.

It is to be understood that the description herein is merely illustrative of the principles of this invention and that various changes and modifications thereof may be made by those skilled in the art within the spirit and scope of the invention as encompassed by the following claims: For example, any implementation for freezing a follicular unit in view, such as freezing air, a spray, a cold tip—etc may be used. Further, although the specification disclosed the use of a camera, any means for capturing the image may be used such as a digital reader.

What is claimed is:

1. Harvesting apparatus for the removal of a follicular unit of hair from an area of a patient for transplant to another area, said apparatus comprising a hollow instrument having a diameter with a cutting edge about equal to the diameter of critical anatomical parts of a follicular unit of hair, said apparatus including a mechanism for aligning said instrument with a selected follicular unit, means for cooling or freezing the skin surface around the selected follicular unit and means for advancing said instrument for cutting around the selected follicular unit.

2. Apparatus as in claim 1 wherein said mechanism for alignment includes video means for viewing the image produced by said system.

3. Apparatus as in claim 2 wherein said mechanism for alignment includes fiber optic means located within said instrument, said fiber optic means being energy coupled to a light source for illuminating a selected follicular unit with which said instrument is aligned.

4. Apparatus as in claim 2 wherein said mechanism for alignment includes a camera for capturing said image and means coupled to said camera for displaying said image on said video means.

5. Apparatus as in claim 1 wherein said instrument has a rotating element attached to a fixture, said mechanism for alignment includes a camera or imaging device located within said fixture, said fixture being fixedly attached to an elongated portion having dimensions for fitting a human hand.

6. Apparatus as in claim 5 wherein said system includes a camera for capturing said image and means coupled to said camera for displaying said image on said video means.

7. Apparatus as in claim 6 wherein said elongated portion has a proximal end, said proximal end having extended there from a chamber portion including said camera, said chamber portion having said hollow instrument axially aligned therewith.

8. Apparatus as in claim 1 wherein said means for cooling or freezing comprises means for introducing a medium in liquid or vapor form to the identified follicular unit.

9. Apparatus as in claim 8 also including suction means for removal of an excised follicular unit.

10. Apparatus as in claim 5 also including means for positioning said elongated portion with respect to a selected follicular unit.

11. A method of harvesting follicular units of hair for implanting, said method comprising the steps of positioning a hollow instrument having a cutting edge with a diameter about equal to the diameter of critical anatomical parts of a follicular unit of hair over a target area, aligning said instrument with the axis of a follicular unit to be excised, cooling or freezing about the follicular unit, inserting said instrument into the scalp or skin about the cooled or frozen skin surface of the follicular unit, advancing said instrument to cut the scalp or skin about the cooled or frozen skin surface of the follicular unit, and removing said follicular unit.

12. A method as in claim 11 wherein said step of aligning comprises capturing an image of said selected follicular unit, displaying said image on a video screen, to facilitate positioning said instrument along the axis of said selected follicular unit.

13. Harvesting apparatus as in claim 1 also including means for killing the shaft of a selected follicular unit.

14. Harvesting apparatus as in claim 13 wherein said means for killing the shaft comprises a needle adapted to deliver an energy source such as electric or ultrasonic or thermogenic or chemical.

15. A method as in claim 11 also including the step of killing the shaft of the frozen selected follicular unit.

16. Apparatus as in claim 4 wherein said means for aligning comprises of robotic subassembly.

17. Apparatus as in claim 1 wherein said system is connected to a robotic arm having extended there from a chamber portion including said camera or a digital reader, said chamber portion having said hollow instrument axially aligned therewith which moves in multiple dimensions for aligning hairs within a follicular unit to position for cutting around said follicular unit.
